(19) United States
(12) Patent Application Publication
(10) Pub. No.: US 2026/0295306 A1
(43) Pub. Date: Oct. 1, 2026

(54) SYSTEMS AND METHODS RELATING TO BODY SCULPTING USING LASER-ENHANCED ACOUSTIC CAVITATION

(71) Applicants: The Regents of the University of Michigan, Ann Arbor, MI (US); University of Kansas, Lawrence, KS (US)

(72) Inventors: Yannis Paulus, Ann Arbor, MI (US); Xueding Wang, Ann Arbor, MI (US); Xinmai Yang, Lawrence, KS (US)

(21) Appl. No.: 19/480,607

(22) PCT Filed: May 1, 2024
(86) PCT No.: PCT/US2024/027256

§ 371 Date: Oct. 31, 2025

Related U.S. Application Data
(60) Provisional application No. 63/499,347, filed on May 1, 2023.

(51) Int. Cl. A61N 7/02; A61N 7/00

(52) U.S. Cl. CPC A61N 7/02; A61N 2007/0008

(57) ABSTRACT

Provided herein are systems and methods that use synchronized laser and ultrasound energy to break up adipose tissue. The systems and method find use for therapeutic and/or cosmetic removal of adipose tissue.

### CROSS REFERENCE TO RELATED APPLICATION

This application claims priority to and the benefit of U.S. Provisional Patent Application Ser. No. 63/499,347 filed May 1, 2023, which is incorporated herein by reference in its entirety and for all purposes.

### GOVERNMENT SUPPORT

This invention was made with government support under EY029489 awarded by the National Institutes of Health. The government has certain rights in the invention.

### FIELD

Provided herein are systems and methods that use synchronized laser and ultrasound energy to break up adipose tissue. The systems and method find use for therapeutic and/or cosmetic removal of adipose tissue.

### BACKGROUND

Body sculpting or contouring is a procedure to reduce or eliminate excessive fat, shape areas of the body, and tighten skin. Many different approaches have been employed, each having drawbacks. Cryolipolysis or coolsculpting uses extreme cold to break down fat cells. The procedure is recommended only for people who are in good general health with no neurologic or orthopedic problems and who are looking for spot fat reduction in specific areas on the body. Cryolipolysis is not recommend for people who have obesity. Multiple sessions may be needed with 6-8 weeks between sessions. Skin surfaces may be irregular after healing. An alternative, laser treatment, works through the application of controlled heat to dismantle subcutaneous fat. Potential risks include infection, skin necrosis, dimpling, lumpiness, numbness, scarring, discoloration or sagging skin. Ultrasonic cavitation, or ultrasound cavitation, uses ultrasonic energy to break apart fat deposits. An advantage of ultrasound cavitation is that it has an excellent safety profile. However, existing ultrasonic cavitation system need improvement in efficiency of fat cell breakup. Improved systems and methods are needed to enhance the safety and efficiency of adipose tissue break up.

### SUMMARY

The present invention, termed a laser-enhanced acoustic cavitation body sculpting (LAC-S), uses synchronized laser and ultrasound to enhance cavitation effect in optical absorptive materials. Experiments conducted during development of the present invention demonstrated that treatment of adipose tissue with LAC-S improved the breakup of fat cells compared to prior existing systems.

In some embodiments, provided herein is an apparatus for performing synchronized laser and ultrasound treatment of adipose tissue. This apparatus may be specially constructed for the desired purposes, and/or it may comprise and integrate general-purpose instruments selectively activated or reconfigured (e.g., by a computer program) to carry out the methods described herein. In some embodiments, the computer program is stored in a non-transitory, tangible computer readable storage medium, or any type of media suitable for storing electronic instructions, which may be coupled to a computer having a processor. Furthermore, any computing systems referred to in the specification may include a single processor or may be architectures employing multiple processor designs for increased computing capability.

In some embodiments, provide herein are methods for the treatment of tissues or cells with synchronized ultrasound and laser.

The systems and methods herein are not limited by type of ultrasound employed. In some embodiments, the ultrasound is a high intensity focused ultrasound (HIFU) system such as an ExAblate 2000 (multielement phased-array transducer, 1.0-1.5 MHz frequency) or a Model JC unit (13.5 cm focal length and 0.8 MHz transducers). In some embodiments, the HIFU includes a piezoelectric ultrasound transducer. More specifically, in some embodiments, the type of transducer used is a concave focusing transducer with a fixed aperture and focal length, a phased array transducer which comprises multiple piston transducers that are arranged on the truncated surface of a spherical bowl, or a flat transducer/fully populated phased array (e.g., Model-JC HIFU system, Chongqing HAIFU™ Company, Chongqing, China). In some embodiments, the HIFU system is guided, assessed, and monitored by a camera, sonography, magnetic resonance imaging (MRI), ultrasound imaging, or magnetic resonance-guided focused ultrasound (MRgFUS). In some embodiments, the mechanical movement of the transducer determines the position of the focal point, with electronic steering of the ultrasound beam allowing fine control of the focal spot location. In some embodiments, the mechanical movement of the HIFU system is controlled by software and/or coupled to a user interface, a transducer, multiple transducers, and/or a display. In some embodiments, the size of the transducer is variable. In some embodiments, the ultrasound is applied through an acoustic window on the target, by external or extracorporeal transducer, or by insertion of the transducer into the target. In some embodiments, the extracorporeal transducer has a wide aperture and long focal length. In some embodiments, the HIFU system operates through a frequency range of 20 KHz to 200 MHz and an amplitude range of 0.5 MPa to 5.5 MPa.

The systems and methods herein are not limited by type of laser employed. In some embodiments, the laser is a pulsed nanosecond laser such as a diode-pumped solid-state (DPSS) type. In some embodiments, the laser can produce approximately a 532 nm wavelength with pulse repetition frequency of approximately 10 Hz and pulse duration of approximately 3-5 ns. In some embodiments, the laser can generate a fixed laser pulse energy of approximately 50 mJ.

The ultrasound and laser systems and methods may be integrated in a number of different ways. In some embodiments, the laser system is triggered by a delay pulse generator (Model DG535, Stanford Research Systems, Sunnyvale, CA, USA). In some embodiments, a delay pulse generator is also used to trigger the HIFU system to temporally synchronize the ultrasound and laser pulses. In some embodiments, the trigger from delay pulse generator is supplied to a function generator (33250A, Agilent Technologies, Santa Clara, CA, US) to produce a 0.5 MHz sine wave burst. In some embodiments, the output burst from the function generator is first amplified by 50 dB through an RF amplifier (2100 L, Electronics & Innovation Ltd, Rochester, NY, US) and then passed to a HIFU transducer through an impedance matching circuit (Impedance Matching Network H107, Sonic Concepts, Bothell, WA, USA). In some embodiments, the HIFU transducer (H-107, Sonic Concepts, Bothell, WA, US) has a central frequency of 0.5 MHz and radius of curvature of 63.2 mm with focal depth and focal width of 21.42 mm and 3.02 mm, respectively. In some embodiments, the target and the HIFU transducer are submerged inside a water tank filled with degassed, deionized water such that the target is at the focal spot of the transducer. The water tank size is variable and selected based on the subject treated and the area of treatment. In some embodiments, the laser beam is focused using a convex lens to result in a beam diameter of about 2 mm on the treatment area on the target. In some embodiments, the laser beam is passed through the hole in the center of the HIFU transducer and is spatially aligned with the HIFU focal region on the target before each treatment. In some embodiments, for the spatial synchronization, the laser induced photoacoustic wave from the target is detected using the 0.5 MHz HIFU transducer. In some embodiments, the wave is acquired on digital oscilloscope (TBS 2000B, Tektronix Inc., Beaverton, OR, USA). In some embodiments, the HIFU transducer is scanned across the target cross-section until the maximum photoacoustic signal is detected, indicating that the focal region of the HIFU transducer overlaid the laser spot on the target. In some embodiments, for the treatment, an ultrasound burst of 1000 cycles is repeated at 10 Hz (2% duty cycle) to minimize the effect of heat during the treatment. In some embodiments, each laser pulse is synchronized at the rarefactional phase of the ultrasound wave. In some embodiments, this is done by first precisely measuring the traveling time of the laser-induced photoacoustic (PA) wave propagating from the target to the HIFU transducer and then the time reversal of this PA wave propagation enables precise phase control when temporally synchronizing the laser pulses with the HIFU bursts. When detecting PA wave produced by 532 nm laser pulses, the produced PA signal was received by the HIFU transducer, and the position 1 in FIG. 1 was connected. In some embodiments, the adipose tissue is positioned for targeting by ultrasound and laser.

The methods herein are not limited by the nature of the condition being treated. In some embodiments, the condition being treated is cellulite or excess adipose tissue. In some embodiments, the condition being treated is cancer or tissue growths. In some embodiments, the condition being treated is inflammation. In some embodiments, the condition being treated is neurodegenerative disease. In some embodiments, the condition being treated is chronic and non-malignant pain.

The methods herein are not limited by the identity or nature of the subject. In some embodiments, the subject being treated is an animal. More specifically, in some embodiments, the subject being treated a cat, dog, cow, pig, chicken, or non-human animal. In some embodiments, the subject being treated is a human. More specifically, in some embodiments, the subject being treated is a male or female. In some embodiments, the subject is an adult. In some embodiments, the subject is a juvenile.

In some embodiments, the systems and methods employ one or more safety features. In some embodiments, the safety features provide for temperature control. In some embodiments, temperature control is managed using a temperature sensor (e.g., thermometer), a timer, and/or a temperature adjustment control mechanism, which can be operated automatically or manually. In some embodiments, the temperature adjustment control mechanism is embodied in software. In some embodiments, then an undesired temperature is detected by a sensor or camera, the temperature adjustment control mechanism alters one or more properties of the system or method, including, but not limited to, treatment cycle timing, on/off status, location of treatment, and the like.

The disclosed methods may further employ pharmacological and/or chemical agents, independently, or in conjunction, with the disclosed invention to treat the subject. In some embodiments, drugs are used in conjunction with the disclosed systems and methods. In some embodiments, a pain relieving drug is used in conjunction with the disclosed systems and methods.

In some embodiments, provided herein is a system that includes an ultrasound generator, a laser, and a controller that synchronizes ultrasound energy with laser energy. In some embodiments, the ultrasound generator is a high intensity focused ultrasound (HIFU) system and the laser is a pulsed nanosecond laser system and the controller is a delay pulse generator. In some embodiments, the laser is configured to generate a fixed laser pulse energy of 50 mJ and the HIFU system is configured to operate through an amplitude range of 0.98 MPa to 2.45 MPa.

In some embodiments, the systems and methods herein comprise: a) a computer processor; b) non-transitory computer memory comprising one or more computer programs and/or a database, wherein the one or more computer programs configured to carry out one or more or all of: controlling the laser, controlling the ultrasound, receiving data from sensors, adjusting parameters based on sensor information, receiving user input, and providing user feedback.

### BRIEF DESCRIPTION OF THE DRAWINGS

FIG. 1 shows a schematic of the experimental setup. Detection of photoacoustic signal occurs at position 1 and HIFU treatment occurs at position 2.

FIGS. 2A-2F show pork belly fat samples after treatment with 9,000 ultrasound and/or laser pulses. FIG. 2A shows a pork belly fat sample after ultrasound-only treatment. FIG. 2B shows a pork belly fat sample after laser-only treatment. FIG. 2C shows a pork belly fat sample after combined ultrasound and laser treatment. FIG. 2D shows a magnified view of the left treated area in FIG. 2C. FIG. 2E shows a magnified view of the right treated area in FIG. 2C. FIG. 2F shows the pork belly fat parameters.

FIG. 3 shows the area of pork belly fat affected after treatment with 9,000 ultrasound and/or laser pulses with: 1) ultrasound PNP of 2.45 MPa; 2) laser pulse energy of 50 mJ; and 3) ultrasound PNP of 2.45 MPa and laser pulse energy of 50 mJ.

FIG. 4 shows the area of pork belly fat removed after treatment with 9,000 combined ultrasound and laser pulses. Laser pulse energy was kept at 50 mJ and ultrasound PNP of 0.49, 0.98, 1.47, 1.96 and 2.45 MPa was used. “*”: p<0.05; “*’: p<0.01; “*”: p<0.001.

FIG. 5 shows the area of pork belly fat removed after treatment with 9,000 combined ultrasound and laser pulses. Ultrasound PNP was fixed at 2.45 MPa and laser pulse energies of 10, 20, 30, 40 and 50 mJ were used. “*”: p<0.05; “*’: p<0.01; “*”: p<0.001.

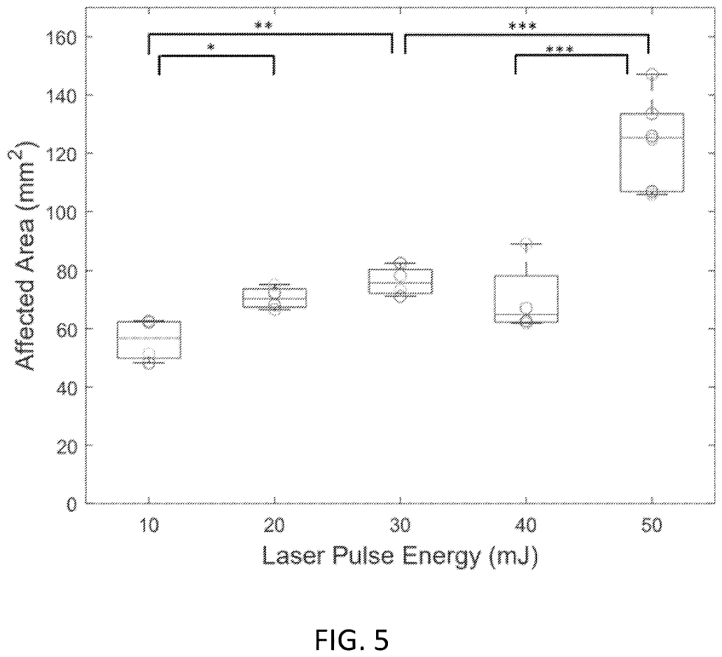

### DETAILED DESCRIPTION

### 1. Definitions

Although any methods, materials, devices, or instruments similar or equivalent to those described herein can be used in the practice or testing of embodiments described herein, some preferred methods, compositions, devices, and materials are described herein. However, before the present systems and methods are described, it is to be understood that this invention is not limited to the particular instruments, devices, software, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a domain” is a reference to one or more domains and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

“Animal”: As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

“Approximately”: As used herein, the term “approximately” and “about” is intended to encompass normal statistical variation as would be understood by those of ordinary skill in the art as appropriate to the relevant context. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Patient”: As used herein, the term “patient” or “subject” refers to a human or any non-human animal (e.g., mouse, rat, rabbit, dog, cat, cattle, swine, sheep, horse or primate) to whom therapy is administered. In many embodiments, a patient is a human.

As used herein, the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), optical discs, and magnetic tape. In certain embodiments, the computer memory and computer processor are part of a non-transitory computer (e.g., in the control unit). In certain embodiments, non-transitory computer readable media is employed, where non-transitory computer-readable media comprises all computer-readable media with the sole exception being a transitory, propagating signal.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks, whether local or distant (e.g., cloud-based).

As used herein, the term “in electronic communication” refers to electrical devices (e.g., computers, processors, etc.) that are configured to communicate with one another through direct or indirect signaling. Likewise, a computer configured to transmit (e.g., through cables, wires, infrared signals, telephone lines, airwaves, etc.) information to another computer or device, is in electronic communication with the other computer or device.

As used herein, the term “transmitting” refers to the movement of information (e.g., data) from one location to another (e.g., from one device to another) using any suitable means.

### Example 1

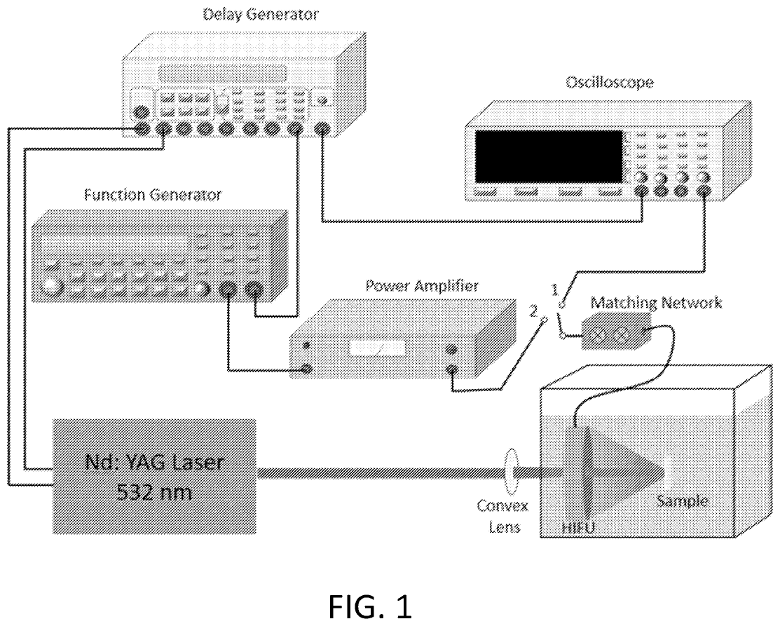

A detailed schematic of an exemplary setup is shown in FIG. 1. The setup is a combination of a high intensity focused ultrasound (HIFU) system and a pulsed nanosecond laser system. The laser system has a 532 nm wavelength (Surelite SL III-10, Continuum, Santa Clara, CA, USA) with pulse repetition frequency of 10 Hz and pulse duration of 3-5 ns. The laser system was triggered by a delay pulse generator (Model DG535, Stanford Research Systems, Sunnyvale, CA, USA). The same delay pulse generator was also used to trigger the HIFU to temporally synchronize the ultrasound and laser pulses. The trigger from delay pulse generator was supplied to a function generator (33250A, Agilent Technologies, Santa Clara, CA, US) to produce a 0.5 MHz sine wave burst. The output burst from the function generator was first amplified by 50 dB through an RF amplifier (2100 L, Electronics & Innovation Ltd, Rochester, NY, US) and then passed to a HIFU transducer through an impedance matching circuit (Impedance Matching Network H107, Sonic Concepts, Bothell, WA, USA). The HIFU transducer (H-107, Sonic Concepts, Bothell, WA, US) has a central frequency of 0.5 MHz and radius of curvature of 63.2 mm with focal depth and focal width of 21.42 mm and 3.02 mm, respectively.

The sample and the HIFU transducer were submerged inside a water tank filled with degassed, deionized water such that the sample was at the focal spot of the transducer. The laser beam was focused using a convex lens to result in a beam diameter of about 2 mm on the treatment area on the sample. The laser beam was passed through the hole in the center of the HIFU transducer and was spatially aligned with the HIFU focal region on the sample before each treatment. For the spatial synchronization, the laser induced photoacoustic wave from the sample was detected using the 0.5 MHz HIFU transducer. The wave was acquired on digital oscilloscope (TBS 2000B, Tektronix Inc., Beaverton, OR, USA). The HIFU transducer was scanned across the sample cross-section until the maximum photoacoustic signal was detected, indicating that the focal region of the HIFU transducer overlaid the laser spot on the target. For the treatment, ultrasound burst of 1000 cycle was repeated at 10 Hz (2% duty cycle) to minimize the effect of heat during the treatment.

Each laser pulse was precisely synchronized at the rarefactional phase of the ultrasound wave. This was done by first precisely measuring the traveling time of the laser-induced photoacoustic (PA) wave propagating from the target to the HIFU transducer. Then the time reversal of this PA wave propagation enabled precise phase control when temporally synchronizing the laser pulses with the HIFU bursts as described previously [9]. When detecting PA wave produced by 532 nm laser pulses, the produced PA signal was received by the HIFU transducer, and position 1 in FIG. 1 was connected.

### Example 2

The treatment was carried out on pork belly fat samples to titrate the ultrasound and laser parameters for lipid removal. The first few pork belly fat samples were treated with high peak negative pressure (PNP) ultrasound-only to find an ultrasound amplitude which did not result in any treatment effect on the sample. It was found that an ultrasound PNP of less than 2.94 MPa did not remove any lipids from the sample. The same experiment, when repeated with laser-only, found laser pulse energy less than 60 mJ (19 mJ/mm² with a focal spot of 2 mm in diameter) to be ineffective in producing any treatment effect on pork belly fat samples. For the next experiments on pork belly fat samples, an ultrasound PNP of less than 2.94 MPa and laser pulse energy of less than 60 mJ was used.

Pork belly fat sample treatment with ultrasound-only, laser-only and combined ultrasound and laser. In the first experiment, the samples were treated with ultrasound-only, laser-only, and combined ultrasound and laser using ultrasound amplitude of 2.45 MPa and laser pulse energy of 50 mJ. The area of the fat tissue being removed from the samples was measured to compare the treatment effect among ultrasound-only, laser-only, and combined ultrasound and laser treatment. Treatment was conducted on two distinct locations separated by at least 7.6 mm using 9,000 ultrasound or laser pulses. To ensure the laser temporal synchronization on the negative phase of ultrasound, the temporal synchronization was changed by 0.5 μs after every 2,250 ultrasound/laser pulses during the combined ultrasound and laser treatment. Six treatments were performed on three samples for combined ultrasound and laser treatment group, and four treatments on two samples for ultrasound-only and laser-only group.

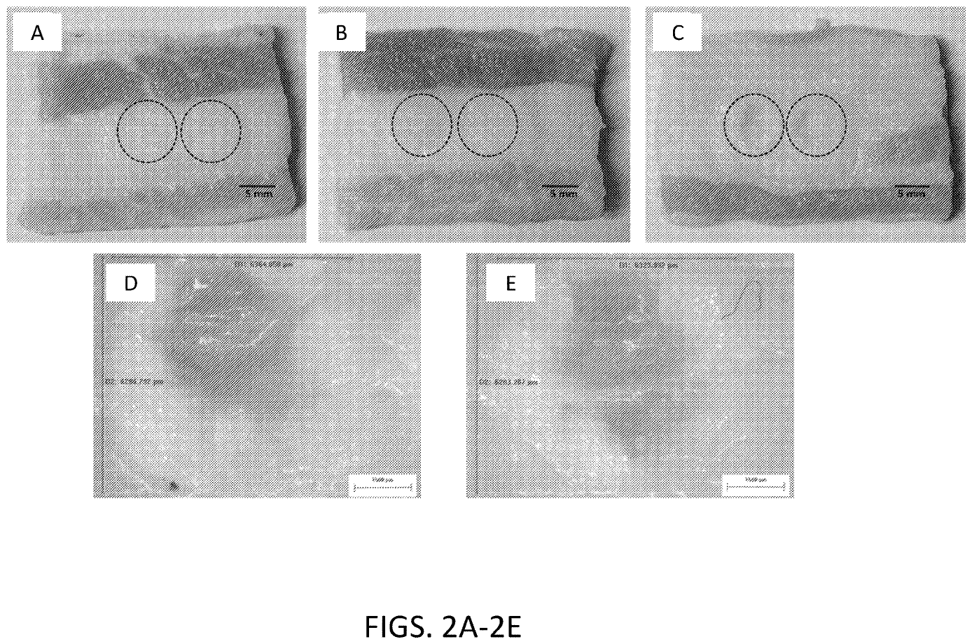

FIG. 2A-2F shows the pork belly fat samples treated with ultrasound-only, laser-only, and combined ultrasound and laser. The ultrasound PNP amplitude of 2.45 MPa and laser pulse energy of 50 mJ (16 mJ/mm²) was used for all the treatments. No treatment effect was visible on samples treated with ultrasound-only (FIG. 2(a)) and laser-only (FIG. 2(b)), whereas, the samples treated with combined ultrasound and laser resulted in fat tissue removal from the samples (FIG. 2(c, d, e)). The removal of fat tissue resulted in cavities on the sample surface. The area of surface cavity after the treatment was used to assess the treatment effect.

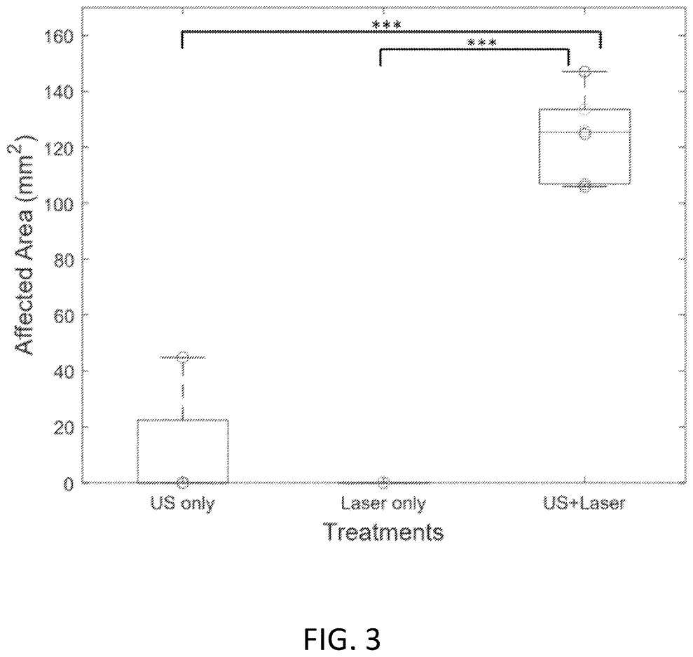

FIG. 3 shows the cavity area on samples after the treatment with ultrasound-only, laser-only, and combined ultrasound and laser. Out of the four treatments carried out on samples using the laser-only, no cavity was observed in any case. For ultrasound-only, out of the four treatments, surface cavity of area 44 mm² was observed in only one treatment. In contrast, for the six treatments conducted using the combined ultrasound and laser, cavities were observed in all treatments with areas ranging from 105 mm² to 147 mm² and a mean cavity area of 124 mm². The treatment area between the combined ultrasound and laser group and ultrasound/laser only group was statistically significant (p<0.001).

Pork belly fat sample treatment with varying ultrasound PNP. In the second experiment, conducted to study the effects of change in ultrasound amplitude and laser pulse energy on the amount of lipid being removed from the pork belly fat sample, the samples were treated with a fixed laser pulse energy of 50 mJ, but with different ultrasound amplitudes of 0.49, 0.98, 1.47, 1.96, and 2.45 MPa. Treatment was conducted on two distinct locations separated by at least 7.6 mm using 9,000 ultrasound or laser pulses. To ensure the laser temporal synchronization on the negative phase of ultrasound, the temporal synchronization was changed by 0.5 μs after every 2,250 ultrasound/laser pulses during the combined ultrasound and laser treatment. Four treatments were performed on two samples for each treatment group.

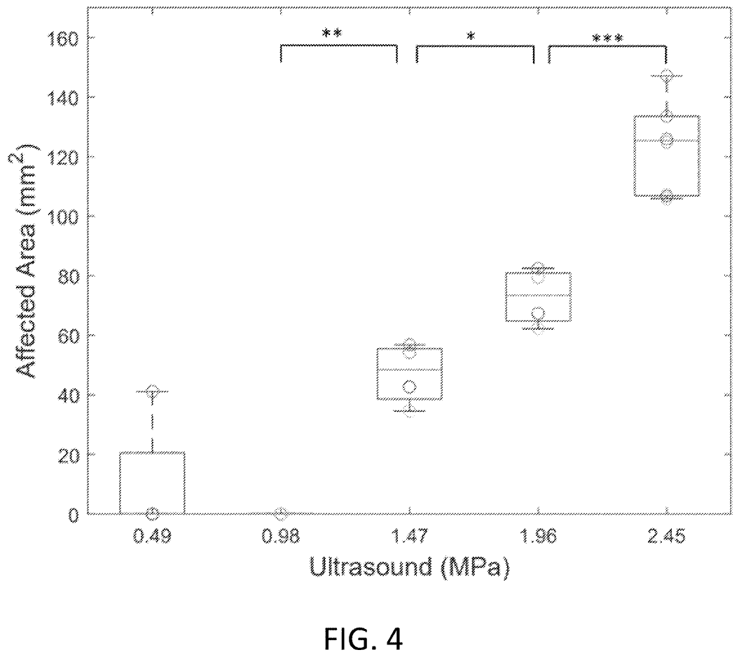

FIG. 4A shows the results of the samples treated with combined ultrasound and laser at a fixed laser pulse energy, but different ultrasound PNPs. The laser pulse energy was fixed at 50 mJ and ultrasound PNPs of 0.49, 0.98, 1.47, 1.96, and 2.45 MPa were used. The mean cavity area of fat tissue removal after treatment generally increased with the increase in ultrasound PNP. The mean cavity areas for 0.49, 0.98, 1.47, 1.96, and 2.45 MPa were 10, 0, 47, 72 and 124 mm², respectively. The areas of fat tissue removal were statistically significant between ultrasound PNP 0.98 MPa and 1.47 MPa (p<0.01), 1.47 MPa and 1.96 MPa (p<0.05), and 1.96 MPa and 2.45 MPa (p<0.001).

Pork belly fat sample treatment with varying laser pulse energy. In the third experiment, conducted to study the effects of change in ultrasound amplitude and laser pulse energy on the amount of lipid being removed from the pork belly fat sample, the samples were treated with a fixed ultrasound amplitude of 2.45 MPa but with different laser pulse energies of 10, 20, 30, 40, and 50 mJ. Treatment was conducted on two distinct locations separated by at least 7.6 mm using 9,000 ultrasound or laser pulses. To ensure the laser temporal synchronization on the negative phase of ultrasound, the temporal synchronization was changed by 0.5 μs after every 2,250 ultrasound/laser pulses during the combined ultrasound and laser treatment. Four treatments were performed on two samples for each treatment group.

FIG. 4B shows the results of the samples treated with combined ultrasound and laser at a fixed ultrasound PNP, but different laser pulse energy levels. The ultrasound PNP was fixed at 2.45 MPa and laser pulse energies of 10, 20, 30, 40, and 50 mJ were used. The mean cavity area of fat tissue removal after treatment generally increased with the increase in laser pulse energy. The mean cavity areas of fat removal for 10, 20, 30, 40, and 50 mJ were 56, 70, 76, 70 and 124 mm², respectively. Statistical significance was achieved for areas of fat tissue removal between 10 mJ and 20 mJ (p<0.05), 40 mJ and 50 mJ (p<0.001), 10 mJ and 30 mJ (p<0.01), and 30 mJ and 50 mJ (p<0.001).

### REFERENCES

1. Moreno-Moraga, J., et al., Body contouring by non-invasive transdermal focused ultrasound. Lasers Surg Med, 2007. 39(4): p. 315-23.
2. Ascher, B., Safety and efficacy of UltraShape Contour I treatments to improve the appearance of body contours: multiple treatments in shorter intervals. Aesthet Surg J, 2010. 30(2): p. 217-24.
3. Coleman, W.P., 3rd, et al., A Multicenter Controlled Study to Evaluate Multiple Treatments With Nonthermal Focused Ultrasound for Noninvasive Fat Reduction. Dermatol Surg, 2017. 43(1): p. 50-57.
4. Weinstein Velez, M., et al., Nonthermal Pulsed Ultrasound Treatment for the Reduction in Abdominal Fat: A Pilot Study. J Clin Aesthet Dermatol, 2018. 11(9): p. 32-36.
5. Coleman, K.M., W.P. Coleman, 3rd, and A. Benchetrit, Non-invasive, external ultrasonic lipolysis. Semin Cutan Med Surg, 2009. 28(4): p. 263-7.
6. Teitelbaum, S.A., et al., Noninvasive body contouring by focused ultrasound: safety and efficacy of the Contour I device in a multicenter, controlled, clinical study. Plast Reconstr Surg, 2007. 120(3): p. 779-789.
7. Qin, Y., et al., The Effect of Laser and Ultrasound Synchronization in Photo-Mediated Ultrasound Therapy. IEEE Trans Biomed Eng, 2020. 67(12): p. 3363-3370.
8. Hazlewood, D. and X. Yang, Enhanced cavitation activity in a slab-shaped optical absorber during photo-mediated ultrasound therapy. Phys Med Biol, 2020. 65(5): p. 055006.
Qin, Y., et al., The Effect of Laser and Ultrasound Synchronization in Photo-MediatedUltrasound Therapy. IEEE transactions on biomedical engineering. 67(12).

What is claimed is:

1. A method comprising: treating adipose tissue with synchronized ultrasound and laser.

2. The method of claim 1, wherein the ultrasound comprises a high intensity focused ultrasound (HIFU) system.

3. The method of claim 1, wherein the laser comprises a pulsed nanosecond laser.

4. The method of claim 1, wherein the ultrasound comprises a high intensity focused ultrasound (HIFU) system and wherein the laser comprises a pulsed nanosecond laser.

5. The method of claim 4, wherein the laser comprises a 532 nm wavelength with pulse repetition frequency of 10 Hz and pulse duration of 3-5 ns.

6. The method of claim 4, wherein the laser is regulated by a delay pulse generator that triggers the laser.

7. The method of claim 4, wherein the HIFU system is regulated by a delay pulse generator that triggers the HIFU system.

8. The method of claim 7, wherein the delay pulse generator is configured to temporally synchronize the ultrasound and laser pulses.

9. The method of claim 8, wherein the delay pulse generator comprises a function generator that produces a wave burst.

10. The method of claim 9, wherein the function generator is configured to generate a 0.5 MHz sine wave burst upon a trigger from the delay pulse generator.

11. The method of claim 10, wherein the function generator comprises a RF amplifier.

12. The method of claim 11, wherein the RF amplifier is configured for an amplification of 50 dB.

13. The method of claim 12, wherein the RF amplifier is configured to amplify a HIFU transducer through an impedance matching circuit.

14. The method of claim 13, wherein the HIFU transducer comprises a central frequency of 0.5 MHz and radius of curvature of 63.2 mm, with a focal depth and focal width of 21.42 mm and 3.02 mm.

15. The method of claim 10, wherein the adipose tissue and the HIFU transducer are submerged inside a water tank that is filled with degassed, deionized water and wherein a focal spot of the HIFU transducer is aimed at the adipose tissue.

16. The method of claim 15, wherein the laser comprises a convex lens.

17. The method of claim 16, wherein the convex lens is configured to focus a laser beam.

18. The method of claim 17, wherein the laser beam has a diameter of 2 mm.

19. The method of claim 18, wherein the laser beam passes through a hole in the center of the HIFU transducer and wherein the HIFU transducer is spatially aligned with a HIFU focal region on the adipose tissue.

20. The method of claim 19, wherein the ultrasound and the laser are configured to spatially align with the HIFU focal region on the adipose tissue.

21. The method of claim 17, wherein the laser beam is configured to induce a photoacoustic wave from the adipose tissue.

22. The method of claim 21, wherein the HIFU transducer is configured to detect the photoacoustic wave from the adipose tissue.

23. The method of claim 22, wherein a digital oscilloscope is configured to acquire the photoacoustic wave from the adipose tissue.

24. The method of claim 23, wherein the HIFU transducer conducts a cross-section scan of the adipose tissue and wherein said scan comprises a duration equal to a minimum time necessary to detect a maximum photoacoustic signal.

25. The method of claim 24, wherein the HIFU transducer further conducts a repetition of 1000 cycles of an ultrasound burst at 10 Hz (2% duty cycle) 26. The method of claim 25, wherein the ultrasound burst of 1000 cycles repeated at 10 Hz (2% duty cycle) is configured to minimize the effect of heat during the treatment.

27. The method of claim 25, wherein each laser pulse is synchronized at a rarefactional phase of each ultrasound wave that is included in each cycle of the ultrasound burst.

28. The method of claim 25, wherein the HIFU transducer and the laser are synchronized such that each laser pulse is achieved by measuring a time of the laser-induced photoacoustic (PA) wave propagating from a target to the HIFU transducer and wherein synchronization of each laser pulse further comprises reversal of said time.

29. A system comprising an ultrasound generator, a laser, and a controller that synchronizes ultrasound energy with laser energy.

30. The system of claim 29, wherein the ultrasound generator comprises a high intensity focused ultrasound (HIFU) system and wherein the laser comprises a pulsed nanosecond laser system and wherein the controller comprises a delay pulse generator.

31. The system of claim 29, wherein the laser is configured to generate a fixed laser pulse energy of 50 mJ and wherein the HIFU system is configured to operate through an amplitude range of 0.98 MPa to 2.45 MPa.

32. The system of claim 29, further comprising adipose tissue positioned for targeting by ultrasound and laser.

33. Use of a system of any of claims 30-32.

34. Use of a system of any of claims 30-32 for the removal of adipose tissue.
